(19) United States
(12) Patent Application Publication
(10) Pub. No.: US 2021/0298703 A1
(43) Pub. Date: Sep. 30, 2021

(54) SELF-DISINFECTING IMAGING SYSTEMS AND METHODS

(71) Applicant: XORAN TECHNOLOGIES LLC, Ann Arbor, MI (US)

(72) Inventors: Jacqueline Vestevich, Ann Arbor, MI (US); Miodrag Rakic, Saline, MI (US); William van Kampen, Saline, MI (US); David Sarment, Saline, MI (US)

(21) Appl. No.: 17/219,237

(22) Filed: Mar. 31, 2021

Related U.S. Application Data
(60) Provisional application No. 63/003,136, filed on Mar. 31, 2020.

(51) Int. Cl. A61B 6/00; A61L 2/10; G06N 20/00

(52) U.S. Cl. CPC A61B 6/4423; A61L 2/10; A61L 2202/14; A61L 2202/24; G06N 20/00

(57) ABSTRACT

An imaging device includes a rotatable gantry having opposing sides, wherein the gantry is rotatable about an axis of rotation, an x-ray source mounted to the gantry, an x-ray detector mounted to the gantry opposite the x-ray source, and one or more ultraviolet light sources mounted to one of the gantry, the x-ray source, and the x-ray detector.

### CROSS-REFERENCE TO RELATED APPLICATIONS

This application incorporates by reference and claims the benefit of priority to U.S. Provisional Application No. 63/003,136 filed Mar. 31, 2020.

### BACKGROUND OF THE INVENTION

This disclosure is generally directed to imaging devices. More specifically, the disclosure relates to an imaging device that is configured to self-disinfect automatically after use.

Imaging systems such as CT scanners are important for evaluating a wide range of medical issues. Keeping the imaging instrument and surrounding space clean is always important, but particularly critical in the event of a viral pandemic such as COVID-19 where CT scanners are used to scan the patient lungs for triage, differential diagnosis, severity assessment, and follow-up monitoring.

In order to be scanned, the patient must physically contact the CT scanner and breathe the air within it. Each patient is exposed to bacteria or viruses living on the surface of the CT scanner and/or in the surrounding air space. During a pandemic, the CT scanner may be used to scan a high volume of potentially infected patients in a short amount of time, increasing the risk of spreading the virus.

Conventional disinfection of a CT scanner includes spraying the surfaces with a disinfectant or wiping the surfaces with antivirus and/or antibacterial cleaning solutions. These processes reduce the time available for operating the CT scanner on patients, and introduce nurses or technicians into a potentially infected area. The methods also consume resources including the cleaning materials and staff time. The cleaning solutions may also cause wear on the equipment.

A need therefore exists for an imaging system that automatically self-disinfects after use.

### BRIEF SUMMARY OF THE INVENTION

To meet the needs provided above and others, the present disclosure provides an imaging system that utilizes ultraviolet (UV) light sources to disinfect the surfaces of the system. The imaging system includes an x-ray source and an x-ray detector mounted onto a gantry, which moves along the length of an operating table on which the patient rests during a scanning procedure. In the illustrated embodiment, the gantry has a vertical ring shape and the x-ray source and detector extend outwardly therefrom parallel to a ground surface opposite to one another on the gantry. One or more UV light sources are embedded within, mounted onto, or otherwise secured to one of the x-ray source, the x-ray detector, and the gantry, and are used to disinfect an interior of the gantry and other imaging device components as well as the patient surfaces such as the operating table, a head holder, a seat, etc.

The UV light source may be an ultraviolet-C (UV-C) light source and/or pulsed xenon UV light. Each UV light source may comprise a UV-C light bulb, a light-emitting diode (LED), or an array of LEDs.

In one embodiment, a first pair of UV light sources is positioned adjacent to opposite sides of the source, and a second pair of UV light sources is positioned adjacent to opposite sides of the detector. The UV light sources may be embedded within or otherwise secured to the gantry, the x-ray source, and the x-ray detector, and may be used in combination with ambient lights embedded in the device or used separately.

The light sources are positioned on the gantry such that the UV light emitted from the UV light sources is directed to an interior region of the ring. Each light source produces an illumination distribution that strikes a portion of the interior surface of the gantry, the operating table, and/or the x-ray source or detector.

The UV light sources and/or additional UV light sources may also emit light outwardly from the gantry into the ambient air. Each UV light source produces an illumination distribution directed away from the imaging system into the surrounding room. In some embodiments, each UV light source may be rotatably mounted to the gantry, the x-ray source, or the x-ray detector such that the light distribution of the UV light source may be rotated in order to disinfect surfaces and ambient air in multiple directions.

During use, the UV light sources may be illuminated automatically during and/or between scans of a patient. The light sources may be illuminated away from the gantry during patient scan, and may be illuminated toward the interior of the gantry between patient scans. The imaging system may include programmable modes of operation based on user preferences.

An objective of the self-disinfecting imaging device is that it quickly and efficiently disinfects surfaces after a patient scan is complete, reducing the exposure of others to dangerous viruses and bacteria.

An advantage of the present invention is that the self-disinfecting imaging device the onboard UV light sources allow for calibration and control of the disinfection sequence to optimize efficacy and effectiveness of disinfection of specific surfaces.

Another advantage of the present invention is that the self-disinfecting imaging device can be used to disinfect surfaces as well as the area surrounding the imaging device.

Additional features and advantages of the disclosed apparatus, system and method are described in, and will be apparent from, the following detailed description and figures. The features and advantages described herein are not all-inclusive and, in particular, many additional features and advantages will be apparent to one of ordinary skill in the art in view of the figures and description. Also, any particular embodiment does not have to have all of the advantages listed herein. Moreover, it should be noted that the language used in the specification has been selected for readability and instructional purposes, and not to limit the scope of the inventive subject matter.

### BRIEF DESCRIPTION OF THE DRAWINGS

The drawing figures depict one or more implementations in accord with the present concepts, by way of example only, not by way of limitations. In the figures, like reference numerals refer to the same or similar elements.

FIG. 1 is a perspective view of an imaging system of the present invention.

FIGS. 2A and 2B are end views of the imaging system of FIG. 1 illustrating interior and exterior illumination distributions, respectively, of the light sources.

FIG. 3 is a side elevation view of the imaging system of FIG. 1 illustrating an interior illumination distribution of the light sources.

FIG. 4 is a perspective view of a further embodiment of an imaging system of the present invention.

FIG. 5 is a side elevation view of the imaging system of FIG. 4.

FIG. 6 is a perspective view of a still further embodiment of an imaging system of the present invention.

FIGS. 7A and 7B are a side elevation views of the imaging system of FIG. 1 illustrating a single layer and a dual layer cover, respectively.

FIG. 8 illustrates a method of use of the imaging systems of FIGS. 1, 4, and 6.

FIG. 9 illustrates a method of quality assurance of the imaging systems of FIGS. 1, 4, and 6.

### DETAILED DESCRIPTION OF THE INVENTION

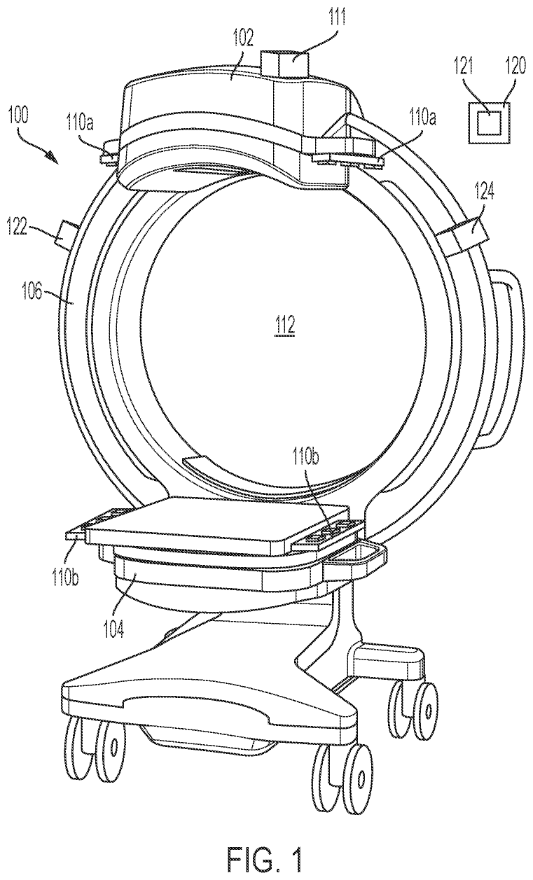

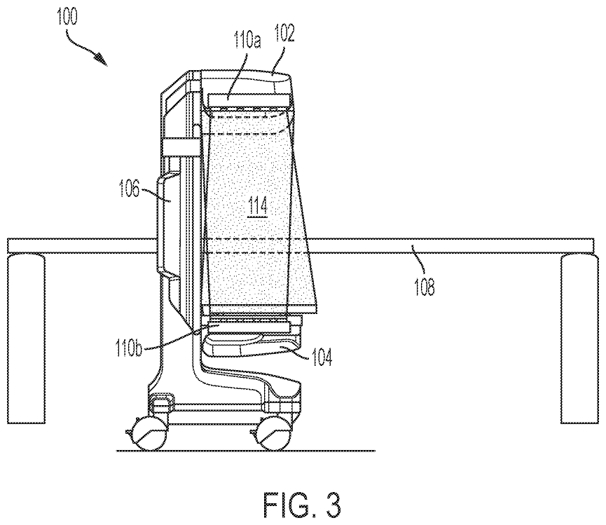

FIGS. 1-3 illustrate an exemplary imaging system 100 of the present application. As shown in FIG. 1, the imaging system 100 includes an x-ray source 102 and an x-ray detector 104 mounted onto a ring-shaped gantry 106, which moves along the length of an operating table 108 on which the patient rests during a scanning procedure, as shown in FIG. 3. The x-ray source 102 and detector 104 are positioned opposite to one another on the vertical gantry 106 and extend outwardly parallel to the floor. In the embodiment illustrated in FIG. 1, ultraviolet (UV) light sources 110 are mounted onto the x-ray source 102 and detector 104 to disinfect the surfaces of the system 100 when illuminated. The UV light sources 110 may be used to disinfect an interior 112 of the gantry 108 and other imaging device components as well as the patient support surfaces such as the operating table 108, a head holder, a seat, etc.

In the embodiment shown in FIGS. 1-3, a first pair 110a of UV light sources 110 is positioned adjacent to opposite sides of the x-ray source 102, and a second pair 110b of UV light sources 110 is positioned adjacent to opposite sides of the x-ray detector 104. The UV light sources 110 may be embedded within or otherwise secured to the x-ray source 102 or detector 104, and may be used in combination with ambient lights embedded in the device 100 or used separately.

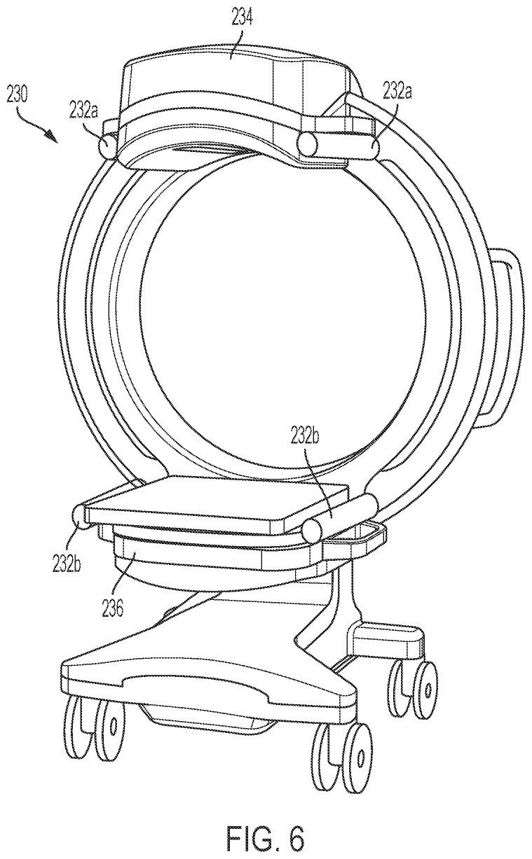

The UV light source 110 may be an ultraviolet-C (UV-C) light source and/or pulsed xenon UV light. Each UV light source 110 may comprise a light-emitting diode (LED) or an array of LEDs. In one embodiment, the UV light source 110 is a plurality of short-wavelength ultraviolet LEDs, ranging from a small number of larger LEDs or a large number of smaller LEDs. In still further embodiments, the UV light source may comprise a UV-C light bulb (see FIG. 6). Other suitable disinfecting light sources may also be used. The light sources 110 are removable and replaceable for easy maintenance.

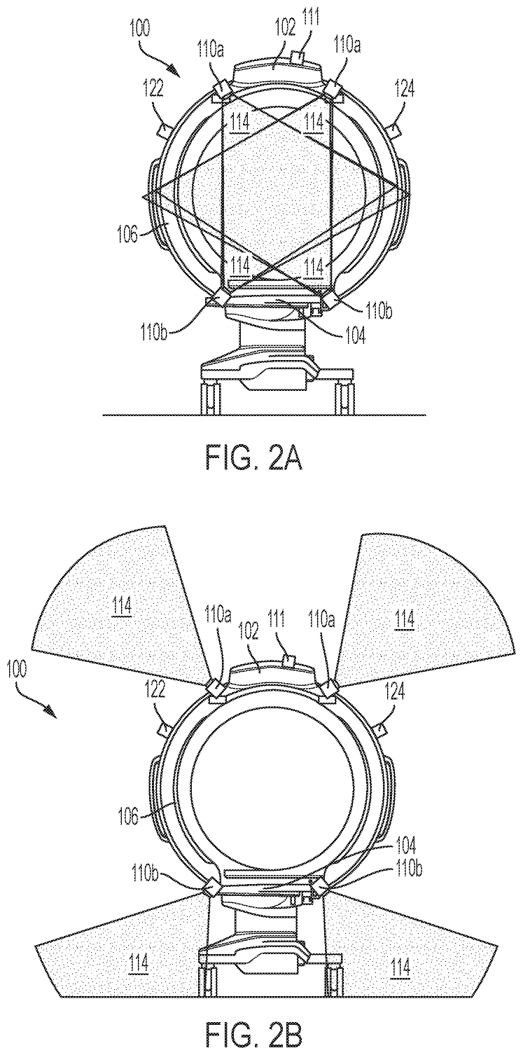

Referring to FIG. 2A, the UV light sources 110 are positioned on the x-ray source 102 or detector 104 such that the UV light distribution 114 emitted from the light sources 110 is directed to an interior region 112 of the gantry 106. In this example, each light source 110 produces an illumination distribution 114 that strikes a portion of the interior surface of the gantry 106, the operating table 108, and/or the x-ray source 102 or detector 104.

In other embodiments, the illumination distribution 114 may have a broader or narrower distribution. In still further embodiments, each UV light source 110 may include a plurality of light elements that are positioned transverse to one another such that illumination of the light source 110 provides light distributions 114 in multiple directions. The imaging system 100 may include a plurality of UV light sources 110 arranged on the x-ray source 102, the x-ray detector 104, or the gantry 106 such that the illumination distributions 114 of individual UV light sources 110 overlaps to ensure that the surfaces of the imaging device 100, operating table 108, and other desired surfaces are disinfected.

As shown in FIG. 2B, the UV light sources 110 may also emit light away from the gantry 108 into the ambient air. In this example, each UV light source 110 produces an illumination distribution 114 directed away from the imaging system 100 into the surrounding room.

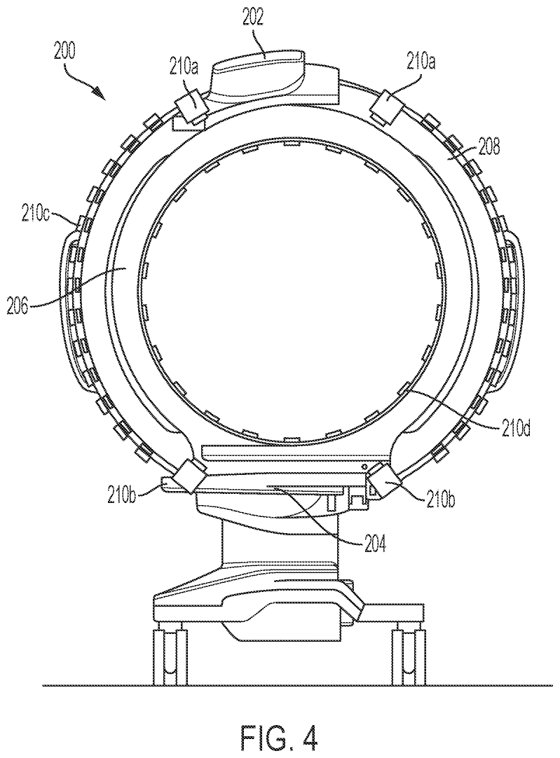

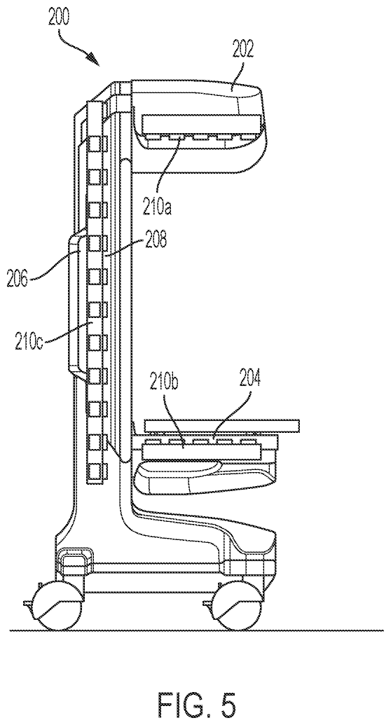

In some embodiments, each UV light source 110 may be rotatably mounted onto the x-ray source 102 or detector 104. The light source 110 may then be rotated along an axis parallel to the side surface of the x-ray source 102 or detector 104 such that rotation of the light source 110 causes the illumination distribution 114 to shift between the interior region 112 of the gantry 106, as shown in FIG. 2A, and outwardly away from the interior region 112 of the gantry 106, as shown in FIG. 2B. In other embodiments, each UV light source 110 may be mounted to the x-ray source 102, the x-ray detector 104, or the gantry 106 through a ball and socket joint, which enables the light source 110 to rotate freely about the connection point in a greater variety of directions. UV light sources 110 may also be mounted on the interior and/or the exterior of the gantry 106 as shown in FIGS. 4 and 5.

Referring to FIG. 3, the gantry 106 is mounted onto a stand 116 to hold the gantry 106 upright. The stand 116 moves along the floor via a plurality of wheels 118. The imaging system 100 is therefore able to move along the length of the operating table 108 as needed. Between patient scans, the UV lights 110 on the imaging system 100 may be illuminated and moved along the length of the operating table 108 for disinfection purposes.

The imaging system 100 may include an indicator light 111 to show that the UV light sources 110 are activated. The indicator light 111 is a non-UV light that is illuminated during the disinfection sequence. While the indicator light 111 is mounted to an outer surface of the gantry 106 as shown in FIGS. 1-3, the indicator light 111 may be positioned in any location that is easy to be seen.

FIGS. 4 and 5 illustrate an alternative embodiment of the imaging device 200 that includes first and second pluralities of UV LED arrays 210d, 210c distributed around an interior surface and an exterior surface, respectively, of the ring-shaped gantry 206 in addition to UV LED arrays 210a, 210b on the x-ray source 202 and detector 204. In the embodiment shown in FIGS. 4 and 5, each of the LED arrays 210c, 210d include a plurality of front-facing LEDs and a plurality of side-facing LEDs. In other embodiments, each of the LED arrays 210c, 210d may include a plurality of front-facing LEDs, a plurality of side-facing LEDs, a plurality of LEDs facing an a still further direction, or a combination thereof.

FIG. 6 illustrates a further alternative embodiment of the imaging device 230 as described with respect to FIGS. 1-3, with the exception of the UV light source 110. Each of the UV light source 232a, 232b of the imaging device 230 is a UV-C light bulb adjacent to opposing side surfaces of the x-ray source 234 and detector 236. In some embodiments, the UV-C light bulb may be used in combination with an array of LEDs.

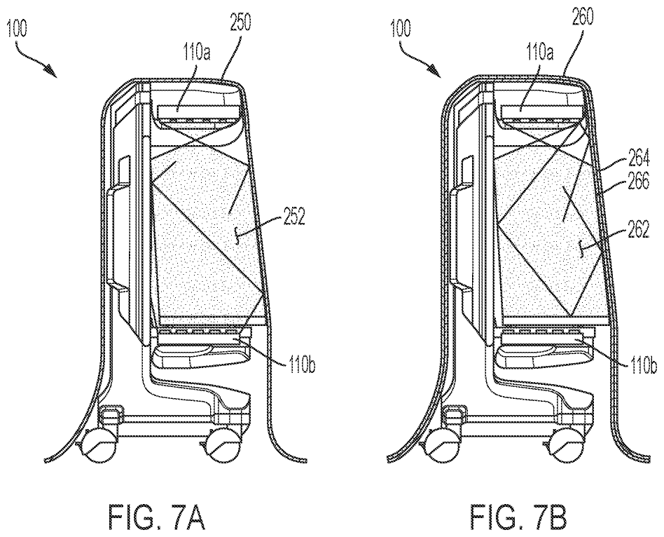

As shown in FIGS. 7A and 7B, in further embodiments, a cover or drape 250, 260 may be positioned over the imaging system 100 to limit exposure of the UV light distribution 252, 262 during a disinfection sequence to patients or other people within the room. During use, the UV light sources 110 may also disinfect the cover or drape 250. In one embodiment, the reflective cover or drape 260 is internally reflective. For example, the drape 250, 260 may be internally reflective such that, when the drape 250, 260 is placed over the imaging system 100 and the UV light sources 110 are in operation, the UV light 252, 262 is reflected within the draped area to concentrate the effect of the UV light in the covered space (i.e., the surfaces of the imaging system 100) rather than allowing the UV light to pass beyond the imaging system 100 towards the ambient environment.

In the example illustrated in FIG. 7A, a reflective cover 250 may be made from reflective material, such as a reflective metallic foil (e.g., a reflective aluminum foil) or an expanded polytetrafluoroethylene. In the example illustrated in FIG. 7B, the reflective cover 260 may be made from a reflective or non-reflective material 264 having an internal surface coated by a reflective coating 266.

The components of the imaging device 100 may be painted with a protective paint on the surfaces subject to UV disinfection to prevent degradation from prolonged and repeated UV exposure.

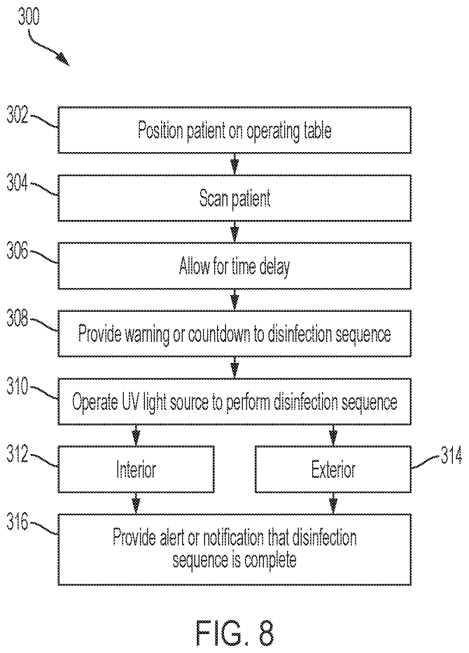

FIG. 8 illustrates a method 300 of use of the imaging device 100, 200. In a first step 302, the patient is positioned on the operating table. The imaging device 100 is then operated in step 304 to scan the patient. In step 306, the imaging device 100 has completed the scan and delays the start of the disinfection for a predetermined period of time. During the time delay, the patient moves from the operation table and the patient and personnel leave the room. The final moments of the predetermined period of time include a warning or countdown that the UV light sources will be illuminated shortly in step 308.

In step 310, the system 100 begins the disinfection sequence. The UV light sources 110 direct the light distribution 114 toward the interior region 112 of the gantry 106 in step 312, and then direct the light distribution 114 away from the interior region 112 of the gantry 106 into the surrounding space in step 314. Once the disinfection sequence is complete, the system 100 triggers a ready indicator in step 316.

Referring to FIG. 1, the imaging system 100 also includes a computer 120 having a processor 121, memory, and storage. The computer 120 is programmed to perform the functions and control the function and operation of the imaging system 100 in the manner described herein. More specifically, the computer 120 controls the performance of a disinfection sequence including the operation of the UV lights 110 to disinfect surfaces and/or ambient air, and may be programmed with modes of operation based on user preferences.

For example, the computer 120 includes configurable parameters such as manual disinfection, automatic disinfection, a time delay after the scan is completed to begin the disinfection sequence, loudness, duration and intensity of the UV disinfection sequences and/or operation of the UV light sources for both the interior and exterior regions, language of audible indications, messages, warnings, countdowns, and other parameters.

The processor 121 may be programmed to automatically capture data. The automatic data capture log may capture information such as implementation dates and times of the disinfection sequence, a log of the parameter settings for each disinfection sequence, efficacy and sufficiency results of each disinfection sequence, the duration of each disinfection sequence, the status of each UV light source, the status and last recoating of surfaces with a UV protective coating, and more. The data capture log also includes a record of dates and times of certain events, such as, but not limited to, the installation and replacement dates and times of the UV light sources, the manufacturing date of the surfaces of the imaging device 100, and the date of repainting of the surfaces with UV protective coating. As the imaging device 100 and the disinfection sequence continues to operate, the imaging device 100 collects operational data to show which surfaces and which ambient air space are disinfected, the efficacy and sufficiency of the disinfection, and the length of time that the disinfection sequence has operated between maintenance events. By tracking and analyzing this data, the system 100 self-identifies and self-reports when UV light sources need maintenance, when surfaces need to be recoated or repainted, and other maintenance measures need to be undertaken. The system 100 may provide notifications to the user when such maintenance measures are due.

For example, the data log captured by the processor may monitor the disinfection of surfaces utilizing a digital camera 124 as described below. The images from the camera 124 indicate whether the surfaces struck by the light distribution 114 of the UV light sources 110 are disinfected. If image from the camera 124 shows that a surface that falls under the light distribution 114 of the UV light source is not disinfected, a notification is sent to the user that maintenance is needed within the area of insufficient disinfection. In some examples, the data log may capture additional details such as the surface is either fully not disinfected or is disinfected but with a lower efficacy rate. If the surface is fully not disinfected, the UV light source needs to be replaced, and the notification sent to the user indicates that the UV light source is due for replacement. If the surface is disinfected at a lower efficacy rate, the duration of the disinfection sequence may need to be extended or the intensity of the UV light source may need to be increased. The notification sent to the user may indicate that such parameters for the disinfection sequence need to be modified to improve the efficacy of the disinfection sequence.

In some embodiments, the imaging system 100 includes an algorithm that processes data and is configured to automatically adjust one or more parameters of the disinfection sequence. More specifically, the processor 121 receives data related to operations of a disinfection sequence, including, for example, an efficacy of a disinfection sequence over a period of time. The processor 121 receives image data from a digital camera or other sensor 124 indicating the percentage of surfaces being disinfected as described below. The processor 121 also tracks operation data such as, but not limited to, the duration of time that the UV-C light sources are activated, the strength of the UV-C light sources, and the rate at which the UV-C light sources move along the length of the operation table.

The algorithm monitors and analyzes the data to identify a trend that the same area of surfaces are being disinfected over time, but that the disinfection rate for a specific duration decreased from 100% disinfection to 98% disinfection after a specific number of disinfection sequences. Based on the collected data, the algorithm determines that emitting light from the UV-C light sources for an additional three seconds will increase the disinfection rate from 98% to 100%. The algorithm provides an output including instructions to adjust the duration of time that the UV-C light sources are activated. The processor 121 then receives the output of the algorithm and adjusts one or more of the operations of an automatic disinfection sequence. In other examples, the algorithm output may include instructions to adjust other parameters of the disinfection sequence. The algorithm may be a machine learning algorithm, a model-based algorithm, or use of a set of premeasured or precomputed calibration data.

One or more machine learning algorithms operating on usage data of the imaging device can be used to control aspects of the system, such as automatically configuring or providing recommendations for configuring the parameters such as, for example, the time delay, the duration, and the intensity of the UV disinfection sequence.

The imaging system 100 may provide alerts to the user, the patient, and other nearby personnel related to the disinfection sequence. The alert may communicate, for example, a disinfection sequence being underway, the presence of ultraviolet emissions, a warning that a disinfection sequence will begin shortly, and a status such as the start or the end of the disinfection sequence. The alert may be visual, text-based, or audible.

A separate handheld controller such as a tablet may be in communication with the processor and include functionality to activate or deactivate the disinfection sequence and to provide notifications to the user that the disinfection is complete or other alerts. In some examples, the handheld controller enables the user to adjust the parameters of the disinfection sequence.

As shown in FIG. 1, a digital camera 124 may be mounted to the gantry 106 to capture images of the UV light sources 110 and/or light distribution 114 during disinfection. The camera 124 may be directed to the interior region 112 of the gantry 106 using a long integration during the disinfection sequence to confirm the extent of the illumination distribution and which surfaces were disinfected.

In some embodiments, a digital camera or other sensor 124 is mounted to the gantry 106 to capture images of the surfaces of the imaging system 100 and operating table 108 after the disinfection sequence is completed. In one embodiment, the digital camera 124 captures and provides images that distinguish between surfaces have been disinfected or radiated using the UV-C light source and surfaces have not been disinfected. In other embodiments, the images captured by the digital camera 124 are processed by the processor 121, which distinguishes between surfaces have been disinfected and surfaces have not been disinfected. The images may be captured during the disinfection sequence and/or after the disinfection sequence is completed. The automatic data capture collects the images and the data derived from the images.

In some embodiments, the images are analyzed and an efficacy and/or sufficiency of a disinfection sequence is derived from the image data. The image data captured by the camera or sensor 124 therefore provides a quality assurance to ensure that the UV light sources are sufficiently disinfecting the surfaces.

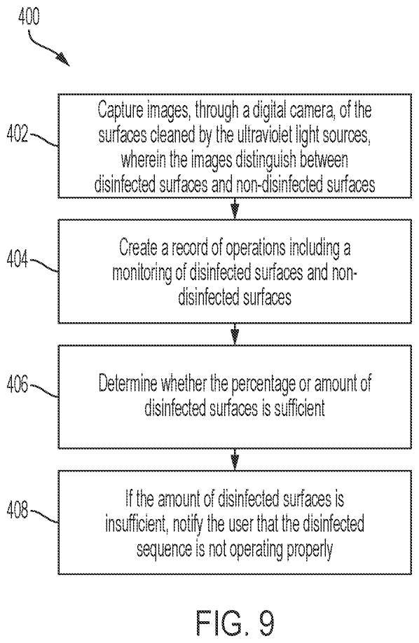

The imaging device 100 may implement the method of quality assurance 400 shown in FIG. 9 utilizing the digital camera 124. In the first step 402, the imaging device 100 captures images distinguishing between disinfected surfaces and non-disinfected surfaces through the digital camera 124. Within the disinfected surfaces, the image data may show a percentage of disinfection of a surface area to indicate an efficacy rate. The processor 121 collects a record or log of the operations, including which surfaces have been disinfected, the efficacy of disinfection of the disinfected surfaces, and which surfaces have not been disinfected in step 404.

In step 406, the processor 121 monitors the record and determines if an insufficient area of the surfaces have been disinfected. In some embodiments, the user defines the minimum threshold amount or percentage of surfaces to be cleaned through a configurable parameters. In other embodiments, the system 100 determines a minimum threshold based on the data capture log and/or other data. In other embodiments, the system 100 identifies surfaces that are typically disinfected and confirms that such surfaces have been disinfected. If the processor 121 determines that an insufficient amount has been cleaned, the processor 121 notifies the user that the disinfection sequence is not operating properly in step 408 in order to enable the user to fix and/or manually clean the imaging device 100. For example, the processor 121 may notify the user that the disinfection sequence is not operating properly by sounding an audible alarm, illuminating a warning light, or automatically locking the operation controls of the imaging device 100.

The user may review the record or log of the operations in order to confirm proper operation of the imaging device. For example, if a UV light source stops working, the camera or other sensor 124 identifies that a portion of the surfaces that are typically disinfected were not disinfected or radiated. The user may immediately take appropriate, corrective action to replace the UV light source and/or manually clean the imaging device 100.

The imaging system 100 may also include an output device that visually and/or audibly communicates a status of operation of the one or more UV light sources 110.

The imaging system 100 may include a motion detector 122 in communication with the processor 121 to detect nearby human movement. If the motion detector 122 detects human movement, a signal or alert is provided to the processor 121 to immediately stop operation of the UV light sources 110 and/or to restart the audio and/or visual warning signals indicating that the disinfection process is underway. In one example, a motion detector 122 may be located on the gantry 106. In other examples, the motion detector 122 is located on a wall of the room in which the imaging system 100 is located or another location remote from the imaging system 100.

Without further elaboration, it is believed that one skilled in the art can use the preceding description to utilize the claimed inventions to their fullest extent. The examples and embodiments disclosed herein are to be construed as merely illustrative and not a limitation of the scope of the present disclosure in any way. It will be apparent to those having skill in the art that changes may be made to the details of the above-described embodiments without departing from the underlying principles discussed. In other words, various modifications and improvements of the embodiments specifically disclosed in the description above are within the scope of the appended claims. For example, any suitable combination of features of the various embodiments described is contemplated.

What is claimed is:

1. An imaging device comprising:
a rotatable gantry having opposing sides, wherein the gantry is rotatable about an axis of rotation;
an x-ray source mounted to the gantry;
an x-ray detector mounted to the gantry opposite the x-ray source; and
one or more ultraviolet light sources mounted to one of the gantry, the x-ray source, and the x-ray detector.

2. The imaging device of claim 1, wherein the one or more ultraviolet light sources are configured to emit light into an interior region of the gantry between the x-ray source and the x-ray detector.

3. The imaging device of claim 1, wherein the one or more ultraviolet light sources are configured to emit light away from an interior region of the gantry between the x-ray source and the x-ray detector.

4. The imaging device of claim 1, wherein the one or more ultraviolet light sources are configured to emit light into an interior region of the gantry and away from the interior region of the gantry.

5. The imaging device of claim 1, wherein the one or more ultraviolet light sources includes a first pair of ultraviolet light sources mounted on opposing sides of the x-ray source and a second pair of ultraviolet light sources mounted on opposing sides of the x-ray detector.

6. The imaging device of claim 1, wherein the one or more ultraviolet light sources are rotatably mounted.

7. The imaging device of claim 1, wherein the one or more ultraviolet light sources comprises short-wavelength ultraviolet light emitting diodes.

8. The imaging device of claim 1, wherein the one or more ultraviolet light sources comprises xenon ultraviolet light.

9. The imaging device of claim 1, further including an output device that audibly and/or visually communicates a status of operation of the one or more UV light source.

10. A method comprising the steps of:
providing an imaging device comprising:
a rotatable gantry having opposing sides, wherein the gantry is rotatable about an axis of rotation;
an x-ray source mounted to the gantry;
an x-ray detector mounted to the gantry opposite the x-ray source;
one or more ultraviolet light sources mounted to one of the gantry, the x-ray source, and the x-ray detector; and
a processor configured to control operation of the one or more ultraviolet light sources;
positioning an operating table in an interior region of the gantry between the x-ray source and the x-ray detector;
operating the imaging device to scan a patient on the operating table;
upon completion of the scan and after a time delay of a predetermined amount of time after completion of the scan, emitting ultraviolet light from the one or more ultraviolet light sources to disinfect at least one or more of the patient support surface, a volume of air between the x-ray source and the x-ray detector, and one or more surfaces of the imaging device.

11. The method of claim 10, wherein the step of emitting ultraviolet light from the one or more ultraviolet light sources further disinfects at least some volume of air or one or more surfaces surrounding the imaging device.

12. The method of claim 10, further comprising the step of providing, through the processor, an alert related to the disinfection sequence.

13. The method of claim 12, wherein the alert is one of visual, text-based, and audible.

14. The method of claim 13, wherein the alert communicates one of a disinfection sequence in operation, a presence of ultraviolet emissions, a warning that a disinfection sequence will begin shortly, a status indicating a start of the disinfection sequence, and a status indicating an end of the disinfection sequence.

15. The method of claim 10, wherein the processor includes one or more of the following configurable parameters: manual disinfection, automatic disinfection, the time delay of a predetermined amount of time after completion of the scan to begin disinfection, loudness, duration and intensity of the ultraviolet disinfection sequence for the interior region, duration and intensity of the ultraviolet disinfection sequence for the exterior region, language of audible indications, messages, warnings, and countdowns.

16. The method of claim 10, wherein the time delay is one of a user-defined amount of time set through configurable parameters and an amount of time determined using an algorithm operating on usage data of the imaging device.

17. The method of claim 10, wherein the processor is configured to:
operate an automatic disinfection sequence in accordance with a set of parameters;
receive an output of an algorithm that processes usage data of the imaging device; and
adjust one or more parameters in the set of parameters of a subsequent automatic disinfection sequence based on the output of the algorithm.

18. The method of claim 17, wherein the algorithm comprises one of a machine learning algorithm, a model-based algorithm, and a calibration data.

19. The method of claim 17, wherein the one or more parameters is a duration that the ultraviolet light is emitted from the one or more ultraviolet light sources.

20. The method of claim 17, wherein the one or more parameters is an output intensity of the one or more ultraviolet light sources.
